(19) United States
(12) Patent Application Publication
(10) Pub. No.: US 2009/0105594 A1
(43) Pub. Date: Apr. 23, 2009

(54) Blood Vessel Finder

(76) Inventors: Connell Reynolds, Fairburn, GA (US); Sridhar Madala, Houston, TX (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/256,927

(22) Filed: Oct. 23, 2008

Related U.S. Application Data
(60) Provisional application No. 60/999,974, filed on Oct. 23, 2007.

(51) Int. Cl. A61B 8/06

(52) U.S. Cl. 600/454

(57) ABSTRACT

Various methods and systems are provided for finding blood vessels. In one embodiment, a method includes extracting a plurality of pulsed wave Doppler data streams, where each data stream corresponds to one of a plurality of depths; determining blood flow information for each of the plurality of depths based on the corresponding data stream, the blood flow information including flow velocity, flow direction, and amplitude of the data stream; and providing a visual display based upon the determined blood flow information, where the visual display includes the determined flow velocity, flow direction, and amplitude of the data stream corresponding to at least one of the plurality of depths.

### CROSS REFERENCE TO RELATED APPLICATIONS

This application claims priority to copending U.S. provisional application entitled “Blood Vessel Finder” having Ser. No. 60/999,974, filed Oct. 23, 2007, the entirety of which is hereby incorporated by reference.

### BACKGROUND

Medical personnel are often charged with the task of injecting a medicine or drug into a patient or drawing blood from a patient. These tasks may be complicated if an appropriate blood vessel is not detected by the medical personnel. The location of a blood vessel may be undetectable for various reasons, including for example, if the patient has very low blood pressure, is obese, or is very young. As a result, locating blood vessels and aligning catheters and/or needles for insertion may require specialized training.

Existing products commonly found in the market place to assist with needle insertion include ultrasound 2-D imaging devices for needle guidance, continuous wave Doppler ultrasound devices for blood flow detection, and optical devices for vein visualization, all of which require specialized training in their use. The Site˜Rite device made by BARD Access Systems uses 2-D ultrasound imaging for needle guidance. Continuous wave Doppler ultrasound devices include MultiDop, FreeDop, and similar devices from Nicolet. The Veinlite device from Translite uses an optical technique to show veins.

### SUMMARY

Briefly described, one embodiment, among others, comprises a method for finding a blood vessel and indicating associated blood vessel information. The method comprises extracting a plurality of pulsed wave Doppler data streams, where each data stream corresponds to one of a plurality of depths; determining blood flow information for each of the plurality of depths based on the corresponding data stream, the blood flow information including flow velocity, flow direction, and amplitude of the data stream; and providing a visual display based upon the determined blood flow information, where the visual display includes the determined flow velocity, flow direction, and amplitude of the data stream corresponding to at least one of the plurality of depths.

Another embodiment, among others, comprises a system for finding a blood vessel and indicating associated blood vessel information. The system comprises means for extracting a plurality of pulsed wave Doppler data streams, where each data stream corresponds to one of a plurality of depths; means for determining blood flow information for each of the plurality of depths based on the corresponding data stream, the blood flow information including flow velocity, flow direction, and amplitude of the data stream; and means for providing a visual display based upon the determined blood flow information, where the visual display includes the determined flow velocity, flow direction, and amplitude of the data stream corresponding to at least one of the plurality of depths.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is an illustration of a blood vessel finding system utilizing ultrasound techniques according to one embodiment of the present disclosure;

FIG. 2 is an illustration of the blood vessel finding system of FIG. 1 including a display unit according to one embodiment of the present disclosure;

FIGS. 3A-3D are graphical representations of various displays of the display unit of FIG. 2 according to one embodiment of the present disclosure;

FIG. 4 is a flow chart illustrating a method for finding a blood vessel and indicating associated blood vessel information using the blood vessel finding system of FIG. 1 according to one embodiment of the present disclosure; and

FIG. 5 is a flow chart illustrating a method for providing audio indication of a blood vessel using the blood vessel finding system of FIG. 1 according to one embodiment of the present disclosure.

### DETAILED DESCRIPTION

Disclosed herein are various embodiments of systems and methods related to locating blood vessels. Reference will now be made in detail to the description of the embodiments as illustrated in the drawings, wherein like reference numbers indicate like parts throughout the several views.

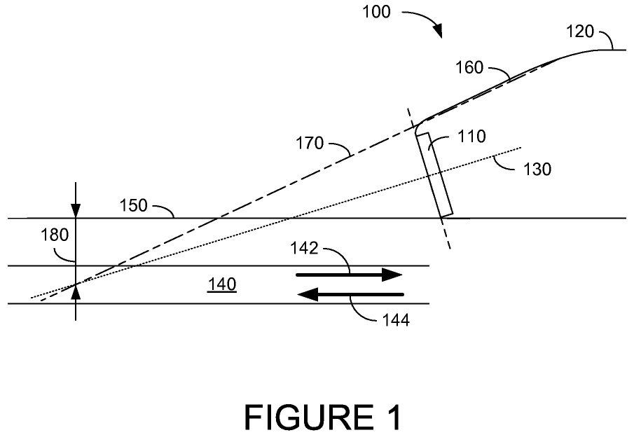

FIG. 1 is an illustration of a blood vessel finding system 100 utilizing Doppler ultrasound techniques according to one embodiment of the present disclosure. In the embodiment of FIG. 1, a crystal ultrasound transducer 110 is embedded in the bottom of a probe unit 120 for ultrasound transmission and/or reception. The probe unit 120 may also include electronics for transmission, reception, demodulation, and digitization. “Diagnostic Ultrasound, Principles and Instruments” (Fifth Edition 1998) by F. K. Kremkau and “Doppler Ultrasound Physics, Instrumentation and Signal Processing” (Second Edition 2000) by D. H. Evans and W. N. McDicken discuss Doppler ultrasound techniques and instrumentation that may be incorporated in the blood vessel finding system 100. The entireties of both the Kremkau and Evans/McDicken references are hereby incorporated by reference.

In one embodiment, the probe electronics implements a single crystal pulsed wave Doppler system. An ultrasound beam is emitted perpendicular to the face of the crystal transducer 110 along path 130. As illustrated in FIG. 1, when positioned over a blood vessel 140 (e.g., artery or vein), the ultrasound beam penetrates the skin surface 150 and intersects with the blood vessel 140. In one embodiment, the crystal transducer 110 is spherically focused with a nominal focal point of about 25 mm. A small amount of ultrasound coupling gel may be located between the skin surface 150 and the transducer face to improve ultrasound transmission and/or reception.

In general, a medical ultrasound unit may operate in a range of about 3-10 MHz in transcutaneous applications. In one embodiment, operating frequency will be in the range of about 5 MHz to 10 MHz. In other embodiments, frequencies above 10 MHz may be used. Higher probe frequencies of about 8 MHz to 10 MHz may be more suitable for shallow vessel detection (e.g., less than about 20 mm in depth) and lower frequencies about 5 MHz to 6 MHz may be more suitable for deeper vessel detection (e.g., more than 20 mm in depth). In one embodiment, among others, different frequencies may be applied by utilizing different probe units 120, each having a crystal transducer 110 with a different frequency.

The reflected ultrasound wave is then detected by the probe unit 120. In one embodiment, the probe unit 120 extracts 32 simultaneous Doppler data streams (e.g., multi-gate Doppler with 32 depth gates). Each resulting stream may then be filtered to exclude wall motion and other artifacts. In a preferred embodiment, the extracting and filtering of data streams is preformed digitally. In other embodiments, the number of Doppler data streams may vary based upon desired depth resolution.

Frequency, amplitude, and phase of the reflected signal may be determined for each data stream using known techniques. For example, “Estimation of Blood Velocities Using Ultrasound, A Signal Processing Approach” (First Edition 1996) by J. A. Jensen discusses Doppler estimation techniques that may be incorporated in the blood vessel finding system 100. The entirety of the Jensen reference is hereby incorporated by reference. The phase change of the signal at each depth is proportional to the mean velocity of blood flow at that depth. The pulsed Doppler implementation also provides direction of blood flow (towards the probe as indicated by arrow 142 and away from the probe as indicated by arrow 144).

In an alternative embodiment, one or more depths may be selected by a user. In one embodiment, only the blood flow information for the data stream(s) at the selected depth(s) and those within a predefined range of the selected depth(s) may be determined. This can improve performance of the blood vessel finding system 100 by reducing the data processing required by the system 100. For example, if the user selects a depth of D, then the blood flow information at depth D is determined from the corresponding Doppler data stream. In addition, only the blood flow information is determined from the two Doppler data streams above and below the selected depth D. Thus, in this example, only five Doppler data streams are processed to determine the corresponding blood flow information. Other ranges about the selected depth D may be predefined and/or selected by the user.

A guide path 160 may also be mounted on top of the probe unit 120 to direct a needle or catheter to desired location. In one embodiment, among others, the guide path 160 may be a groove in which a needle rests. As illustrated in the embodiment of FIG. 1, the guide path 160 design is such that the ultrasound beam intersects needle path 170 at a predefined depth 180 below the skin surface 150. In some embodiments, the guide path 160 may be adjustable to different predefined depths or within a range of depths. In other embodiments, multiple guide paths 160 may be provided, each with a different predefined depth 180.

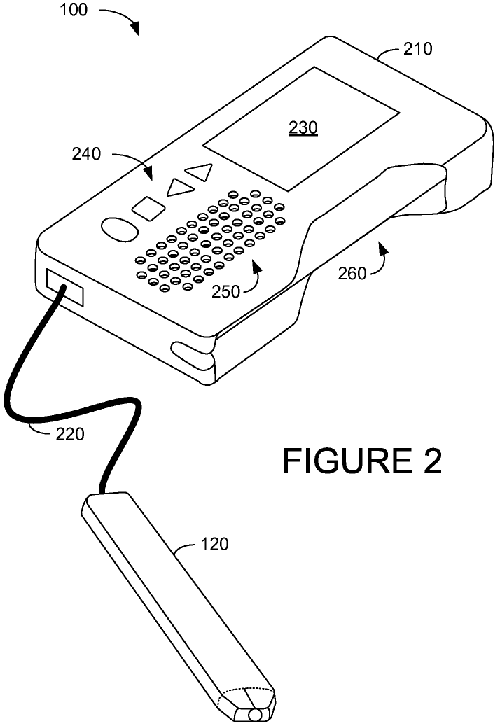

FIG. 2 is an illustration of the blood vessel finding system 100 of FIG. 1 including a display unit 210 according to one embodiment of the present disclosure. In the embodiment of FIG. 2, the display unit 210 and the probe unit 120 are communicatively coupled through a communication cable 220. In another embodiment, the display unit 210 and the probe unit 120 may be communicatively coupled by wireless means such as, but not limited to blue tooth, infrared or other radio-frequency communication means.

The display unit 210 includes a high speed digital signal processor for computation of blood flow velocities, visual displays, and for audio signal generation. In a preferred embodiment, the display unit 210 includes a display 230, keys 240 for user interaction, a speaker for audio output 250. In the embodiment of FIG. 2, the keys 240 on the keypad include: (a) on/off, (b) flip, (c) volume up and (d) volume down. Other buttons may be added optionally for ancillary functions.

Different frequencies may be used by connecting different probe units 120, each having a crystal transducer 110 with a different frequency, to the display unit 210. In the embodiment of FIG. 2, the connected probe unit 120 may be disconnected from the communication cable 220 and replaced by another probe unit with a different frequency. When communicatively coupled by wireless means, a different probe unit 120 may be synchronized and/or connected to the display unit 210 by pressing a key 240 to initiate a synchronization and/or connection process. Similarly, different nominal focal points may be utilized by changing the connected probe unit 120.

In the embodiment of FIG. 2, the display unit 210 also includes a parking/resting place 260 to hold the probe unit 120 when not in use. The display unit 210 may also include provisions for internal battery power and a connector for optional external wall mounted electrical power source. In other embodiments, display unit 210 may have an audio output connection for optional use of an external audio output device such as, but not limited to, larger external speakers, ear phones, or headsets. In alternative embodiments, the probe unit 120 and display unit 210 may be integrated into a single unit.

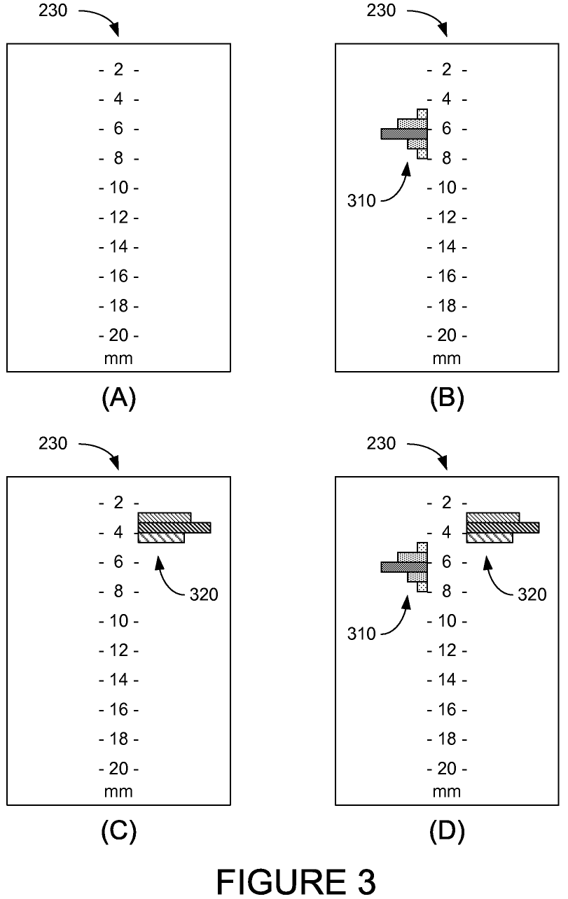

In one embodiment, the display 230 includes a depth scale drawn vertically from top (less depth) to bottom (more depth) in the center of the screen. FIGS. 3A-3D are graphical representations of various displays 230 of the display unit 210 of FIG. 2 according to one embodiment of the present disclosure. In the embodiments of FIGS. 3A-3D, the scale is in millimeters (e.g., a range from about 2 mm to about 20 mm) with a predefined resolution (e.g., a resolution of ⅔ mm). In other embodiments, the scale and range may be different. In some embodiments, the scale and resolution may be selectable by the user. Blood flow information determined from the extracted Doppler data streams may be displayed based upon the resolution of the display 230 and depth scale.

In exemplary embodiment of FIG. 3A, no blood flow has been detected. In some embodiments, blood flow is not detected until one or more predefined criteria are satisfied (e.g., blood flow above a predefined threshold). As blood flow is detected at different depths, a bar whose length is proportional to the mean velocity of the blood flow at that depth will be drawn on the screen. FIGS. 3B-3D provide examples of detected blood flows. Flow away from the probe unit 120 (see arrow 144 of FIG. 1) will be drawn as a horizontal bar from the center scale to the left in a first color and/or pattern. In the embodiment of FIG. 3B, horizontal bars 310 indicate that flow away from the probe unit 120 has been detected at about 5-8 mm in depth and with the highest velocity at just over 6 mm in depth. If the probe unit 120 had been oriented on a person's arm with the crystal transducer 110 directed up the arm from the hand toward the elbow, then this would indicate veinous flow. In a preferred embodiment, the first color is blue.

Flow towards the probe unit 120 (see arrow 142 of FIG. 1) will be drawn as a horizontal bar from the center scale to the right in a second color and/or pattern. In the embodiment of FIG. 3C, horizontal bars 320 indicate that flow towards the probe unit 120 has been detected at about 3-5 mm in depth with the highest velocity at just under 4 mm in depth. If the probe unit 120 had been oriented on a person's arm with the crystal transducer 110 directed up the arm from the hand toward the elbow, then this would indicate arterial flow. In a preferred embodiment, the second color is red.

The intensity of the Doppler signal (i.e., the amplitude of the signal as opposed to the phase) is used to change the hue and/or intensity of the first and second colors and/or patterns. Thus, in one exemplary embodiment, a strong signal of blood flowing away from the probe at a specific depth will be displayed as a deep blue bar to the left. In the embodiment of FIG. 3D, horizontal bars 310 and 320 indicate that blood flow both away from and towards the probe unit 120 has been detected. In both groups of horizontal bars (310 and 320), the signal with the highest intensity is centered in the group. In the exemplary embodiments of FIGS. 3B-3D, the intensity is indicated by the boldness of the horizontal bar patterns.

In alternative embodiments, a simple LED bar graph with one LED per depth (e.g., per millimeter of depth) may be used to convey the depth information to the user in a compact and a cost effective manner. In one embodiment, the intensity of the light of each LED is proportional to Doppler shift. In some embodiments, each led will be a bicolor device (e.g., red/blue). In other embodiments, a 2 dimensional array of LEDs, with a row at predefined depths, may be used to indicate velocity based upon the number of elements that are lit. Additionally, the LED intensity may be varied to indicate the amplitude of the signal associated with the row of LEDs.

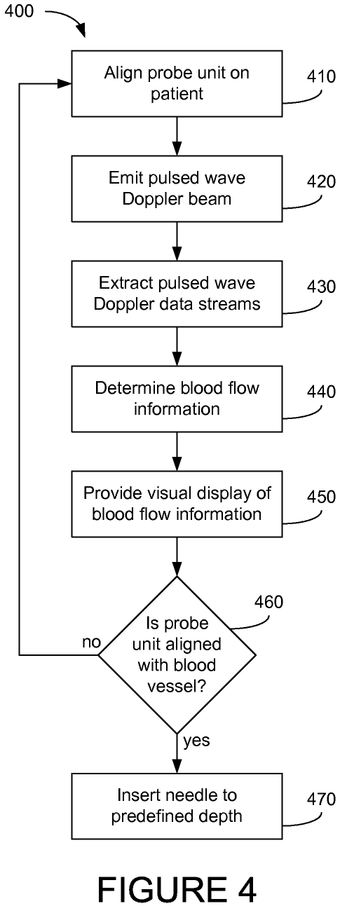

FIG. 4 is a flow chart 400 illustrating a method for finding a blood vessel and indicating associated blood vessel information using the blood vessel finding system 100 of FIG. 1 according to one embodiment of the present disclosure. In block 410, a probe unit 120 is aligned on a patient. For example, the probe unit 120 may be placed over the inside of a patient's forearm. The probe unit 120 emits a pulsed wave Doppler beam in block 420 and extracts a plurality of pulsed wave Doppler data streams in block 430. Each data stream corresponds to one of a plurality of depths within the patient's arm.

Blood flow information including flow velocity, flow direction, and amplitude of the data stream signal is determined based on the corresponding data stream in block 440. In some embodiments, blood flow information for each of the plurality of depths is determined. In other embodiments, only the blood flow information of a limited set of depths is determined. A visual display based upon the determined blood flow information is provided in block 450. In some embodiments, the display 230 of FIG. 3 may be used.

If visual display indicates that the probe unit 120 is properly aligned with a blood vessel (block 460), then a needle may be inserted using the guide path 160 for proper location of the needle at the predefined depth 180 below the skin surface 150 (see FIG. 1). For example, in the embodiment of FIG. 1, when the visual display indicates an adequate blood flow and signal intensity at the predefined depth 180 as determined by the user, then the user may insert a needle by moving along guide path 160 until the predefined depth 180 is reached (block 470). The probe unit 120 may then be removed from the patient's arm in block 480. If the probe unit 120 is not properly aligned, then return to block 410 and the user realigns the probe unit 120.

The blood vessel finding system 100 may also present Doppler audio information to a user based upon depth and blood flow. For example, the user may select a specific depth for audio output. The system may then provide the blood flow signal from that depth gate as a single audio signal as the audio output that is broadcast by speaker 250 (FIG. 2) and/or external audio output device. In other embodiments, audio information from all depths may be provided to the listener by adding the audio signal from each depth to produce a single audio output, which is sent to speaker 250 or other audio output device. As the signal(s) of interest is only present at a few depths, the signal of interest may be obscured by the noise that is summed from all depths.

In an alternate embodiment, an algorithm may be utilized to provide the flow information at multiple specified depths as an audio output. For example, if a signal at a specified depth, or within a specified range of depths, has sufficient amplitude and phase based upon predefined thresholds (e.g., if either or both amplitude and phase are above the predefined thresholds), then audio signals from that depth are included in a combined (multi-depth) audio output stream to the speakers. In this exemplary embodiment, signals which do not meet the threshold criteria may be considered to be noise and, as such, are not be included in the final audio output. In some embodiments, only signals associated with blood flow in a user defined direction may be indicated.

The persistence of the signal over time may also be used as a weighting factor when mixing the signals from multiple gates. In one embodiment, a signal must persist past a predefined threshold (e.g., a few hundred milliseconds) before it is added to the audio output signal. In other embodiments, the longer the signal persists, the larger the weighting factor assigned to that signal. Weighting factors may vary over time in either a linear or exponential manner. In some embodiments, the weighting factors may be normalized. The weighting factor would reset when fails to meet the defined threshold criteria. This inherently rejects noisy signals that are present for only short durations.

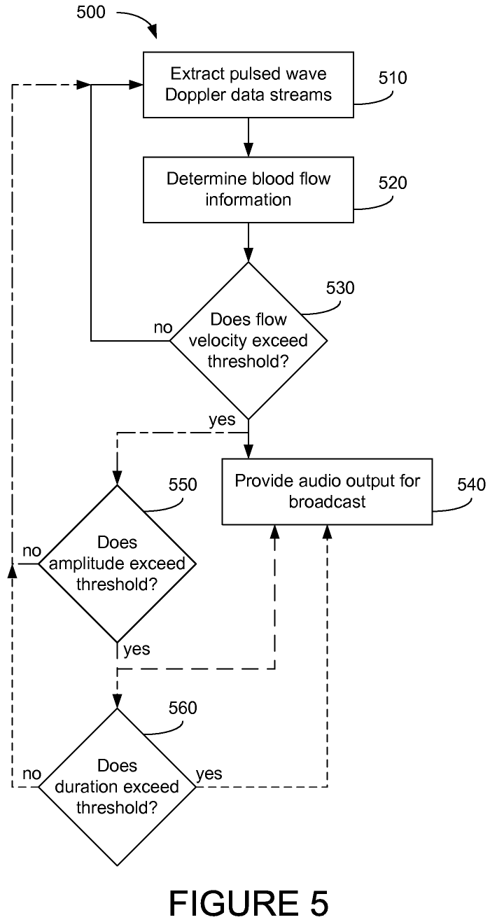

FIG. 5 is a flow chart 500 illustrating a method for providing audio indication of a blood vessel using the blood vessel finding system 100 of FIG. 1 according to one embodiment of the present disclosure. In block 510, a plurality of pulsed wave Doppler data streams, each corresponding to one of a plurality of depths, are extracted. Blood flow information including flow velocity and amplitude of the data stream signal is determined at a user specified depth (or range of depths) based on the corresponding data stream in block 520. In one embodiment, if the determined flow velocity exceeds a predefined threshold (block 530), then an audio output including an audio signal corresponding to the flow velocity is provided for broadcast in block 540. In one embodiment, the audio signal is the blood flow signal (e.g., data stream phase or flow velocity) from the associated depth gate. If the determined flow velocity does not exceed the predefined threshold, then the next set of Doppler data streams are extracted (block 510) and considered.

In an alternative embodiment, if the determined flow velocity exceeds a predefined threshold (block 530), then the amplitude of the data stream is compared to a second predefined threshold in block 550. If the amplitude of the data stream exceeds the second predefined threshold, then an audio output including an audio signal corresponding to the flow velocity is provided for broadcast in block 540. In another embodiment, the duration of time one or both of the flow velocity and/or the amplitude of the data stream exceed the first and/or second threshold, respectively, is compared to a corresponding threshold in block 560. If the duration exceeds the corresponding threshold, then an audio output is provided for broadcast in block 540.

In the case where multiple depths have been selected by the user, the criteria for each depth are evaluated to determine which audio signals corresponding to the selected depths should be included in the audio output. In some embodiments, the audio signals are weighted before combining to form the audio output. The weighting may be based upon the amount of time or duration that the corresponding flow velocity and/or the amplitude of the data stream exceeds its corresponding threshold.

Any process descriptions or blocks in flow charts should be understood as representing modules, segments, or portions of code which include one or more instructions executable by a processor for implementing specific logical functions or steps in the process, and alternate implementations are included within the scope of the preferred embodiment of the present disclosure in which functions may be executed out of order from that shown or discussed, including substantially concurrently or in reverse order, depending on the functionality involved, as would be understood by those reasonably skilled in the art of the present disclosure.

The determination and display of blood flow information of certain embodiments of the present disclosure can be implemented in hardware, software, firmware, or a combination thereof. In the preferred embodiment(s), the determination of blood flow information is implemented in firmware or software that is stored in a memory and that is executed by a suitable instruction execution system. If implemented in hardware, as in an alternative embodiment, the determination of blood flow information can be implemented with any or a combination of the following technologies, which are all well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

The modules, segments, or portions of code, which comprise an ordered listing of executable instructions for implementing logical functions, can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can contain, store, communicate, or transport the program for use by or in connection with the instruction execution system, apparatus, or device. The computer-readable medium can be, for example but not limited to, an electronic, magnetic, optical, electromagnetic, infrared, or semiconductor system, apparatus, or device. More specific examples (a nonexhaustive list) of the computer-readable medium would include the following: a portable computer diskette (magnetic), a random access memory (RAM) (electronic), a read-only memory (ROM) (electronic), an erasable programmable read-only memory (EPROM or Flash memory) (electronic), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory. In addition, the scope of the certain embodiments of the present disclosure includes embodying the functionality of the preferred embodiments of the present disclosure in logic embodied in hardware or software-configured mediums.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, at least the following is claimed:

1. A method for finding a blood vessel and indicating associated blood vessel information, comprising:
extracting a plurality of pulsed wave Doppler data streams, where each data stream corresponds to one of a plurality of depths;
determining blood flow information for each of the plurality of depths based on the corresponding data stream, the blood flow information including flow velocity, flow direction, and amplitude of the data stream; and
providing a visual display based upon the determined blood flow information, where the visual display includes the determined flow velocity, flow direction, and amplitude of the data stream corresponding to at least one of the plurality of depths.

2. The method of claim 1, wherein the flow velocity and flow direction is displayed as a single horizontal bar, where displayed horizontal bar position corresponds to depth and displayed horizontal bar length corresponds to the flow velocity.

3. The method of claim 2, wherein displayed horizontal bar color corresponds to the flow direction.

4. The method of claim 2, wherein displayed horizontal bar pattern corresponds to the flow direction.

5. The method of claim 2, wherein displayed horizontal bar intensity corresponds to the amplitude of the data stream.

6. The method of claim 2, wherein the displayed horizontal bar is provided by a row of LEDs.

7. The method of claim 1, wherein the at least one of the plurality of depths is a user defined depth.

8. The method of claim 1, wherein the plurality of depths comprises a user defined depth and a predefined number of adjacent depths above and below the user defined depth.

9. The method of claim 7, further comprising providing an audio output including an audio signal associated with the at least one of the plurality of depths when the corresponding flow velocity exceeds a predefined threshold.

10. The method of claim 9, wherein the audio signal is not included in the audio output until the corresponding flow velocity exceeds the predefined threshold for a predetermined period of time.

11. The method of claim 10, wherein the audio output includes a plurality of audio signals, each of the plurality of audio signals associated with at least one of the plurality of depths, where each of the plurality of audio signals is weighted based upon the duration of time the corresponding flow velocity exceeds a corresponding predefined threshold.

12. The method of claim 9, wherein the audio signal is not included in the audio output if the corresponding amplitude of the data stream does not exceeds a second predefined threshold.

13. A system for finding a blood vessel and indicating associated blood vessel information, comprising:
means for extracting a plurality of pulsed wave Doppler data streams, where each data stream corresponds to one of a plurality of depths;
means for determining blood flow information for each of the plurality of depths based on the corresponding data stream, the blood flow information including flow velocity, flow direction, and amplitude of the data stream; and
means for providing a visual display based upon the determined blood flow information, where the visual display includes the determined flow velocity, flow direction, and amplitude of the data stream corresponding to at least one of the plurality of depths.

14. The system of claim 13, wherein the flow velocity and flow direction is displayed as a single horizontal bar, where displayed horizontal bar position corresponds to depth and displayed horizontal bar length corresponds to the flow velocity.

15. The system of claim 14, wherein displayed horizontal bar color corresponds to the flow direction.

16. The method of claim 14, wherein displayed horizontal bar intensity corresponds to the amplitude of the data stream.

17. The system of claim 13, further comprising means for providing an audio output including an audio signal associated with the at least one of the plurality of depths when the corresponding flow velocity exceeds a predefined threshold.

18. The system of claim 13, further comprising means for guiding a needle along a predefined path.

19. A system for finding a blood vessel, comprising:
a probe unit configured to transmit a pulsed wave Doppler beam and extract a plurality of pulsed wave Doppler data streams, where each data stream corresponds to one of a plurality of depths; and
a display unit configured to determine blood flow information for each of the plurality of depths based on the corresponding data stream and visually display the determined blood flow information corresponding to at least one of the plurality of depths, where the blood flow information includes flow velocity, flow direction, and amplitude of the data stream.

20. The system of claim 19, wherein the probe unit includes a needle guide path.
